(19) United States
(12) Patent Application Publication
(10) Pub. No.: US 2026/0256840 A1
(43) Pub. Date: Sep. 3, 2026

(54) MUTATED T CELL RECEPTOR BETA CHAIN

(71) Applicant: The Regents of the University of California, Oakland, CA (US)

(72) Inventors: Justin Eyquem, San Francisco, CA (US); Christopher Chang, San Francisco, CA (US)

(21) Appl. No.: 18/875,278

(22) PCT Filed: Jun. 14, 2023
(86) PCT No.: PCT/US2023/068423

§ 371 Date: Dec. 16, 2024

Related U.S. Application Data
(60) Provisional application No. 63/352,057, filed on Jun. 14, 2022.

(51) Int. Cl. A61K 35/17; A61K 40/11; A61K 40/32; A61K 40/42; C07K 14/705; C12N 5/00; C12N 5/0783; C12N 15/10; G01N 33/569; G01N 33/68

(52) U.S. Cl. CPC A61K 35/17; A61K 40/11; A61K 40/32; A61K 40/421; C07K 14/7051; C12N 5/0087; C12N 5/0636; C12N 15/1037; G01N 33/56972; G01N 33/6854; C12N 2510/00; G01N 2333/7051

(57) ABSTRACT

The disclosure provides compositions and methods for generating a population of engineered immune cells, e.g., T cells, that express recombinant or synthetic T cell receptors and are depleted of cells that express endogenous T cell receptors. The disclosure further provides therapeutic methods employing such populations.

### CROSS-REFERENCE TO RELATED APPLICATIONS

This application is a U.S. National Stage of PCT/US2023/068423, International Filing Date Jun. 14, 2023, which claims priority benefit of U.S. Provisional Application No. 63/352,057, filed Jun. 14, 2022, which is incorporated by reference for all purposes.

### REFERENCE TO AN ELECTRONIC SEQUENCE LISTING

The contents of the electronic sequence listing (081906-1382297-247210PC_SL.xml; Size: 4169 bytes; and Date of Creation: Oct. 30, 2023) is herein incorporated by reference in its entirety.

### BACKGROUND OF THE INVENTION

Recombinant TCRs and synthetic receptors with TCR-like architecture (such as HLA-Independent TCRs [HIT]) (see, e.g., Mansilla-Soto, Eyquem et al., Nature Med 28:345-352, 2022, WO2019157454) can be used to redirect immune cells against tumors. In order to use engineered T cells in allogenic applications, T cells expressing an endogenous TCR must be removed to avoid graft-vs-host activity. Antibody-based negative selection (which frequently uses the BW242/412 anti-TCR antibody) is the optimal method for removing cells which express an endogenous TCR. However, this technique does not distinguish between endogenous TCRs, recombinant TCRs, and synthetic receptors with TCR-like architectures due to a shared BW242/412 binding epitope in the human TCR beta constant chain (Kierkels et al., Mol Ther Methods C/in Dev 22:388-400, 2021; published Jun. 23, 2021). Although previous studies have established that BW242/412 does not bind to receptors with a murine TCR beta constant domain, substituting individual residues from mouse TCR beta constant chain to human TCR beta constant chain is not sufficient to remove the BW242/412 epitope from human TCRs (Kierkels et al, supra).

### BRIEF SUMMARY OF ASPECTS OF THE DISCLOSURE

Provided here are compositions and methods in which a T-Cell Receptor (TCR) beta chain constant region is mutated to remove one or more antibody binding epitopes. Thus, in one aspect, the disclosure provides a nucleic acid encoding a human TCR beta chain polypeptide comprising a mutated constant region having at least 90% sequence identity to any one of SEQ ID NOS:1, 2, or 3, wherein the constant region comprises a substitution at a position corresponding to position D111, wherein the substitution is D111K, D111T, D111Q, or 111A; or the constant region comprises a deletion at the position corresponding to D111, wherein the substitution or deletion inhibits binding of antibody BW242/412 to human TCRαβ. In some embodiments, the substitution or deletion abrogates binding of the beta chain polypeptide to antibody BW242/412. In some embodiments, the substitution is D111K. In some embodiments, the substitution is D111T or D111Q. In some embodiments, the mutated constant region comprises a deletion at the position corresponding to D111. In some embodiments, the mutated constant region further comprises a substitution at at least one position corresponding to a position selected from G101, W108, P115, Q118, C130, and G131, wherein the substitution at G101 is Q, D, or N; the substitution at position 108 is A, R, M or L; the substitution at P115 is K, R, or D; the substitution at position 130 is V, E, L or I; and the substitution at position 131 is N. In some embodiments, the mutated constant region has at least 90% identity to SEQ ID NO: 1. In some embodiments, the mutated constant region has at least 90% identity to SEQ ID NO:2. In some embodiments, the mutated constant region has at least 90% identity to SEQ ID NO: 3. In some embodiments, the mutated constant region has at least 95% identity to SEQ ID NO: 1. In some embodiments, the mutated constant region has at least 95% identity to SEQ ID NO:2. In some embodiments, the mutated constant region has at least 95% identity to SEQ ID NO:3. In some embodiments the human TCR beta chain is comprised by an HLA-independent TCR. In some embodiments, the human TCR beta chain is comprised by an HLA-dependent TCR.

In some embodiments, a human TCR beta chain polypeptide comprising a mutated constant region having at least 90% sequence identity, or at least 95% identity, to any one of SEQ ID NOS:1, 2, or 3, wherein the constant region comprises at least one substitution at a position corresponding to G101, D111, or P115. Any amino acid may be substituted for the native amino acid at the position to modulate, e.g., decrease, antibody binding, e.g., BW242/412 binding, to the beta chain. In some embodiments, the substitution at position G101 is any residue other than A. In some embodiments, the substitution at position D111 is any residue other than E or G. In some embodiments, the substitution at position P115 is any amino acid other than A.

In a further aspect, the disclosure provided a nucleic acid encoding a human TCR beta chain polypeptide comprising a mutated constant region having at least 90% sequence identity to any one of SEQ ID NOS:1, 2, or 3, wherein the constant region comprises at least one substitution at a position corresponding to position G101, W108, P115, Q118, C130, and G131, wherein the substitution at G101 is Q, D, or N; the substitution at position 108 is A, R, M or L; the substitution at P115 is K, R, or D; the substitution at position 130 is V, E, L or I; and the substitution at position 131 is N, wherein the substitution reduces binding of an antibody BW242/412 to the mutated constant region. In some embodiments, the nucleic acid comprises at least two substitutions in the constant region. In some embodiments, the mutated constant region has at least 95% identity to any one of SEQ ID NOS:1, 2, or 3. In some embodiments, the mutated constant region binds to an antibody BW242/412 with a binding affinity at least 100× weaker than the antibody binds to any one of SEQ ID NOS:1, 2, or 3.

In a further aspect, the disclosure provides a host cell comprising a nucleic acid encoding a human TCR beta chain polypeptide comprising a mutated constant region as described herein, e.g., in the preceding paragraphs in this section of the application. In some embodiments, the disclosure provides a host cell, e.g., a T cell, a human embryonic stem cell, or a pluripotent stem cell comprising a nucleic acid encoding a human T cell receptor beta chain polypeptide comprising a mutated constant region as described herein, e.g., in the preceding paragraphs in this section of the application. In some embodiments, the host cell is a T cell, e.g, a cytotoxic T lymphocyte, a regulatory T cell, or a Natural Killer T (NKT) cell. In some embodiments, the host cell is engineered to express a transgenic TCR-alpha chain. In some embodiments, the host cell is edited to disrupt expression of endogenous TCR-alpha chain. In some instances, the transgenic TCR-alpha chain is introduced into the host cell at the TRAC locus and disrupts expression of endogenous TCR-alpha chain.

In an additional aspect, the disclosure provides a population of T cells comprising a nucleic acid encoding a mutated human TCR beta chain constant region as described herein. In some embodiments, the population of T cells has less than 1% that express an endogenous TCR. In other aspects, the disclosure provides a method of treating cancer comprising administering to a subject, a population of T cells comprising a mutated TCR beta china as described herein, e.g., a population of T cells as described in this paragraph.

In other aspects, the disclosure provides a method of determining the amount of mis-pairing between endogenous TCR beta chain and a transgenic T cell receptor alpha chain expressed by a population of T cells comprising a variant TCR beta chain comprising a mutated constant region as described herein, the method comprising determining the level of binding of an antibody to individual T cells in the population. In some embodiments, the transgenic T cell receptor alpha chain is edited to disrupt expression of endogenous TCR-alpha chain. In some instances, the transgenic TCR-alpha chain is introduced into the T cell at the TRAC locus and disrupts expression of the endogenous TCR-alpha chain. In many embodiments, the method further comprises removing T cells that bind the antibody from the population.

In a further aspect, the disclosure provides a library of cells that express mutated TCR beta constant regions wherein the library comprises cells comprising TCR different mutations of the TCR beta constant region that span the constant region domain. In some embodiments, the cells are T cells edited at the human TRAC locus to express different mutations of the TCR beta constant region.

### BRIEF DESCRIPTION OF THE DRAWINGS

FIG. 1 depicts a screening strategy to identify mutations in an epitope that abrogate binding of any antibody to the epitope.

FIG. 2 depicts the relative abundance of mutants in a BW242/412-bound fraction (pop 2) vs. the abundance in the HIT+BW242/412-unbound fraction (pop 4).

FIG. 3 provides data showing the enrichment of mutant in BW242/412-Unbound, HIT+ fraction (pop 4) relative to library baseline (pop1) by location in TRBC.

FIG. 4 shows the enrichment of mutants at D111 in BW242/412-Unbound, HIT+ fraction (pop 4) relative to library baseline (pop 1).

FIG. 5 provides data showing that D111K exhibits minimal BW242/412 binding.

FIG. 6 shows calculation of recovery of mutant receptors.

FIG. 7 provides an example of receptor recovery for 2 HIT receptors (wild-type TCR beta chain and D111K mutated beta chain.

FIG. 8 provides data showing recovery rates for various mutations at TCR beta chain positions 111, 115, and 101.

FIG. 9 provides data illustrating that a NY-ESO D111K TCR exhibits markedly reduced anti-TCR antibody binding.

FIG. 10 provides data showing that purification of a T cell population using negative selection resulted in a population of T cells that lack an endogenous TCR, but retain the NY-ESO transgenic TCR, where less than 1% of T cells express an endogenous tCR and about 2% express a mispaired TCR.

FIG. 11: Sequencing results from site-saturation-mutagenesic. Cells transduced with HIT TRBC site saturation mutagenesis libraries were sorted into two populations based on BW242/412 binding. Next generation sequencing was then used to quantify the relative abundance of individual mutations in sorted populations.

FIG. 12: T cells were transduced to express HIT receptors with 5 different amino acids at TRBC D111. BW242/412 binding was then quantifying by measuring the mean fluorescence intensity (MFI) of HIT+ cells stained with PE-conjugated BW242/412.

FIG. 13: Primary human T cells were edited at either TRAC or TRAC and TRBC via CRISPR/Cas-9 and engineered to express either an unmodified TCR beta constant domain (NY-ESO WT TRBC) or a TCR beta constant domain with the D111K mutation (NY-ESO TRBC D111K) from the TRAC locus. Edited cells were then stained with FITC-conjugated anti-TCR antibody (BW242/412) and PE-conjugated NY-ESO dextramer and analyzed via flow cytometry.

FIG. 14: Primary human T cells were edited at TRAC via CRISPR/Cas9 and transduced to express a MART-1 transgenic TCR with either an unmodified TCR beta constant domain (MART-1 WT TRBC) or a TCR beta constant domain with the D111K mutation (MART-1 TRBC D111K) from the TRAC locus. Edited cells were then stained with FITC-conjugated anti-TCR antibody (BW242/412) and APC-conjugated MART-1 dextramer and analyzed via flow cytometry.

### DETAILED DESCRIPTION OF THE DISCLOSURE

### Terminology

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” as used herein refers to the usual error range for the respective value readily known to the skilled person in this technical field. For example, for K_{D} and IC₅₀ values±20%, ±10%, or ±5%, are within the intended meaning of the recited value.

The terms “T-cell” and “T-lymphocyte” as used herein are interchangeable. Examples of T cells include, but are not limited to, memory T cells, regulatory T cells, effector T cells, natural kill T cells, or T cells derived in vitro from embryonic or pluripotent stem cells.

A T-cell receptor (TCR) is a disulfide-linked heterodimeric protein having two variable chains expressed as a part of a complex with invariant CD3 chain molecules. Broadly, each chain of a TCR comprises two extracellular domains: a variable region and a constant region. The constant region is proximal to the cell membrane, followed by a transmembrane domain and a short cytoplasmic tail (i.e., an intracellular domain). The chains of a TCR must assemble before the receptor complex is trafficked to the surface. The variable region of both chains each has three complementarity determining regions (CDRs). Naturally-occurring TCRs are of two types, alpha-beta, which comprise an alpha chain (TCRα) and a beta chain (TCRβ); and gamma-delta, which have a gamma chain (TCRγ) and delta chain (TCRδ). In the context of the present application, a T cell receptor (TCR) generally refers to native and variant TCRs comprising a beta chain (TCRβ or TCRb) unless otherwise specified. Each variable region comprises three CDRs (Complementarity Determining Regions) embedded in a framework sequence, one being the hypervariable region named CDR3. A TCR may bind to an antigen in an HLA-dependent manner or HLA-independent manner. In some embodiments, a variable region of a TCR may bind to a target extracellular antigen in an HLA-independent manner. Such TCRs are designated HLA-independent TCR (HIT).

In humans, there are two TCRβ constant regions genes, TCRB1 and TCRB2, which are very similar not only in sequence but also in genomic organization. Illustrative TCRB1 and TCRB2 polypeptide sequences are available under UniProtKB accession number P10850 and A0A5B9, respectively. The reference TCRB1 and TCRB2 polypeptide sequences provided in the UniProtKB entries are provided in SEQ ID NO:2 and SEQ ID NO:3, respectively. A “human TCRβ” as used herein refers to any allelic form encoded by a human TCRB1 or TCRB2 gene.

A “variant” TCRβ polypeptide as used herein, refers to a R chain comprising a constant region that comprises one or more mutations that reduces, and in some embodiments eliminates, antibody binding, e.g., binding of a BW242/412 antibody, to the beta chain polypeptide. “Engineered TCR” and “mutant TCR” are used synonymously herein and generally mean a TCR having one or more mutations introduced relative to a parental TCR. An engineered TCR may bind to an antigen in an HLA-dependent or HLA-independent manner.

An “HLA-independent TCR” as used herein is a TCR that can recognize an antigen independent of HLA restriction. In an exemplary embodiment, an HLA-independent TCR may bind to an antigen on the cell surface that is not presented by the HLA complex.

As used herein, the term “antibody” means an isolated or recombinant binding agent that comprises the necessary variable region sequences to specifically bind an antigenic epitope. Therefore, an “antibody” as used herein is any form of antibody of any class or subclass or fragment thereof that exhibits the desired biological activity, e.g., binding a specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

As used herein, “affinity” with respect to antibody binding to an epitope generally refers to the strength of the interaction of the antibody with the epitope. In the present disclosure, the affinity of antibody for binding to the target polypeptide is represented by the equilibrium dissociation constant (K_{D}). K_{D} is inversely related to the binding affinity.

The phrase “specifically (or selectively) binds” to a target or “specifically (or selectively) immunoreactive with,” when referring to a protein or peptide, refers to a binding reaction whereby an epitope binds to an antibody or other binding partner. In the context of this invention, the epitope binds to an antibody or target with a K_{D} that is at least 100-fold greater than its affinity for other antigens.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a TCRβ polypeptide “corresponds to” an amino acid in the polypeptide of SEQ ID NO:1 when the residue aligns with the constant region amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or oligonucleotide, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

### TCR Beta Chain Constant Region Mutations

The present disclosure provides TCR beta chain sequence variants that are mutated to remove an epitope commonly recognized by antibodies that bind to wildtype TCR beta 1 or TCR beta 2 constant region genes. Thus, cells expressing a wildtype TCR beta constant chain can be removed from a population of immune cells that are engineered to express a T cell receptor polypeptide comprising the variant, e.g., to generate allogeneic T cell populations for therapy, such as adaptive cell transfer therapies. In some embodiments, such mutated cells that comprises a TCR-targeted CAR or HIT can be used to deplete host T cells while avoiding fratricide killing. TCRs of the present disclosure thus comprise at least one substitution in the constant region. Accordingly, the TCR may have one, two, three, four, or five or more mutations in the beta chain. As understood by one of skill in the art, although the variant TCR beta constant region engineered to remove an epitope of an antibody that binds to wildtype TCR beta 1 or beta 2 constant regions, variants are selected that do not hinder TCR binding to an antigen, in either an HLA-dependent or HLA-independent manner. Furthermore, such a variant TCR beta constant region does not affect TCR signaling, sensitivity to antigen density, and/or overall antitumor activity. In some embodiments, a TCR comprising a mutated TCR beta chain constant region as described herein has at least 90% or at least 95% of the TCR signaling activity, sensitivity to antigen density, and/or overall antitumor activity compared to the corresponding wildtype TCR beta constant region.

Mutations in the TCR beta chain constant region described herein disrupt binding of an antibody, BW242/412 (available from Miltenyi Biosciences), to human TCRαβ. BW242/412 is characterized in EP403156B1 (see, heavy and light chain sequence as listed for clone BMA031, which sequences are incorporated by reference). The TCR beta chain constant region mutations provided by the present disclosure, e.g., D111 mutations, also disrupt binding of an anti TCRα/P antibody, IP26, available from Biolegend. Three other known antibodies, WT31 and T10B9.1A-31 (ThermoFisher Scientific), as well as REA652 (Miltenyi Biosciences), also bind to epitopes that overlap completely with that of BW242/412. Thus, the TCR beta chain constant region mutations, e.g., D111 mutations, also disrupt binding of these antibodies to human TCRαβ.

Thus, in one aspect, described herein is a nucleic acid encoding a human TCR beta chain polypeptide comprising a mutated constant region having at least 90% sequence identity, or at least 95% identity, to any one of SEQ ID NOS:1, 2, or 3, wherein the constant region comprises a substitution at position D111 as determined with reference to SEQ ID NO:1, wherein the substitution is D111K, D111T, D111Q, or D111A. In some embodiments, the substitution is D111K. In some embodiments, the substitution is D111R. In some embodiments, the substitution is D111H. In some embodiments, any amino acid other than E or G may be substituted for D at position 111. In some embodiments the substitution is any one of L, I, V, Y, S, M, F, Q, C, or H. In some embodiments, the residue corresponding to position 111 is deleted. In some embodiments, the mutated constant region has at least 95% identity to SEQ ID NO:1. In some embodiments, the mutated constant region has at least 96% identity, at least 97% identity, at least 98% identity or at least 99% identity to SEQ ID NO:1.

In a further aspect, the disclosure provides a nucleic acid encoding a human TCR beta chain polypeptide comprising a mutated constant region having at least 90% sequence identity, or at least 95% identity, to any one of SEQ ID NOS:1, 2, or 3, wherein the mutated constant region comprises at least one substitution at a position selected from G101, W108, P115, Q118, C130, and G131. In some embodiments, the at least one substitution is at position G101. In further embodiments, the substitution at G101 is Q, D, or N. In some embodiments, any amino acid may be substituted for G at positions 101. In some embodiments, the substituted amino acid at G101 is any amino acid other than A. In some embodiments, the substitution at G101 is any one of T, K, R, M, Z, V, F, I, Y, H, L, W, E, S, C, or P. In some embodiments, the residue corresponding to position 101 is deleted. In some embodiments, the at least one substitution is at position W108. In further embodiments, the substitution at position W108 is A, R, M or L. In some embodiments, the at least one substitution is at position P115. In some embodiments, any amino acid may be substituted for P at positions 115. In some embodiments, the substituted amino acid at P115 is any amino acid other than A. In some embodiments, the substitution is any one of W, L, T, C, I, Y, V, M, A, F, G, H, S, E, or N. In further embodiments, the substitution at position P115 is K, R, or D. In some embodiments, the residue corresponding to position 115 is deleted. In some embodiments, the at least one substitution is at position C130. In further embodiments, the substitution at position C130 is V, E, L or I. In some embodiments, the at least one substitution is at position G131. In further embodiments, the substitution at position G131 is N. In some embodiments, the mutated constant region has at least 95% identity to SEQ ID NO: 1. In some embodiments, the mutated constant region has at least 96% identity, at least 97% identity, at least 98% identity or at least 99% identity to SEQ ID NO:1. In some embodiments, a substitution as described in the present paragraph is combined with a second substitution in in the TCR beta constant region chain, with the proviso that the second substitution is not D111G.

In some embodiments, the human TCR beta chain polypeptide comprises a mutated constant region having at least two mutations as described herein, e.g., as described in the preceding two paragraphs, that decrease, e.g., abrogates binding of an antibody, e.g., antibody BW242/412, to the TCR beta chain polypeptide. In some embodiments, the constant region comprises a mutation at a position corresponding to D111 of any one of SEQ ID NOS:1, 2, or 3, wherein the D at position 111 is replaced with Q or T; or position D111 is deleted, and at least a second mutation. In some embodiments, the D at position 111 is replaced with any one of L, I, V, Y, S, F, Q, or C. In some embodiments, the second mutation is a substitution at position G101, W108, P115, Q118, C130, and G131. In some embodiments, the substitution at G101 is Q, D, or N; or any one of T, K, R, M, Q, V, F, I, Y, H, L, W, E, S, C, or P. In some embodiments, the substitution at position W108 is A, R, M or L. In some embodiments, the substitution at position P115 is K, R, or D; or any one of W, L, T, C, I, Y, V, M, A, F, G, H, S, E, or N. In some embodiments, the substitution at position C130 is V, E, L or I. In some embodiments, the substitution at position G131 is N. In some embodiments, a variant TCR beta constant region chain comprises at least three mutations as described in the present paragraph. In some embodiments, a variant TCR beta constant region chain comprises at least four or at least five mutations as described in this paragraph. In some embodiments, the human TCR beta chain polypeptide comprises at least two mutations as described herein at two of the three residues D111, G101, or P115.

### Evaluation of Antibody-Binding Activity of Variant Beta Chain Constant Regions

A variant beta chain constant regions is considered to “abrogate” binding of an antibody, e.g. BW242/412, to an epitope on a beta chain constant region when binding is reduced by at least 10-fold, at least 50-fold or at least 100-fold compared to binding of the antibody to a wildtype control beta chain that comprises the epitope under the same experimental conditions at physiological conditions suitable for humans. In some embodiments, an antibody exhibits reduced binding to the mutated receptor by at least a 1000-fold compared to control. In some embodiments, a variant beta chain constant region results in reduction of antibody-bound cells in a population of cells by at least 10-fold.

In some embodiments, a mutation, or a combination of mutations, to the TCR beta chain that disrupt binding of an antibody, e.g., BW 242/412, is considered to be useful when, following negative selection performed on a population of T cells comprising cells that express wildtype TCR beta chain and mutated TCR beta chain with the antibody that binds wildtype beta chain, e.g., BW242/412, greater than 10% of the cells recovered after negative selection express the mutant beta chain. In some embodiments, greater than 50% of cells that are recovered following negative selection, harbor a mutation, e.g., a D111K mutation, that disrupts binding of an antibody, e.g., BW 242/412 to a wildtype beta 1 or beta 2 chain. In other words, 10% of cells with a mutant receptor are not bound by antibody, while less than 2% or less than 1% of cells expressing an endogenous TCR beta chain are not bound by antibody. For example, in engineering cells to produce a HIT receptor, less than 1% of cells that express an unmutated HIT receptor may be recovered following negative selection, whereas 50% or greater of cells with a HIT D111K mutation are retained in the cell population following negative selection. As used herein, “negative selection” refers to removing wildtype TCR beta chain-expressing cells from a population of cells, e.g. using an antibody that binds to wildtype TCR beta 1 or beta 2.

In some embodiments, an assay to assess binding of a variant to a TCR beta constant region to an antibody can be performed in a solution having a physiological pH, e.g., pH 7.4 and physiological salt condition, e.g., 150 mM NaCl. In an exemplary assay, binding of antibody, e.g., BW242/412, to cells can be performed at 4° C. for 15 minutes. Cells can then be washed and the level of antibody binding to cells determined, for example using flow cytometry. Cells that do not bind antibody, i.e., “antibody unbound” cells” can be defined as cells with antibody binding equivalent to that of an isotype control. Under these conditions, <2% of T cells with an endogenous TCR should be antibody unbound. Conversely, at least 10% of T cells with a receptor bearing a useful mutation should be antibody unbound. The following example, further describes such an assay:

Mutation performance is assessed by quantifying the fraction of T cells with a given receptor that are recovered following TCR-based negative selection. In this illustrative assay, T cells are engineered to express a mutant beta chain receptor from the TRAC locus via CRISPR/Cas editing. Receptor expression in the edited population of T cells will be quantified via flow cytometry. A known number of T cells will be purified using a TCR-based negative selection kit (e.g., a CliniMACS® TCRα/β Kit employing BW242/412 available from Miltenyi Biotec). The number of cells recovered from negative selection is quantified and receptor expression in the negatively-selected population is further quantified. via flow cytometry. Receptor recovery is assessed by determining the percentage of receptor-positive cells recovered following negative selection.

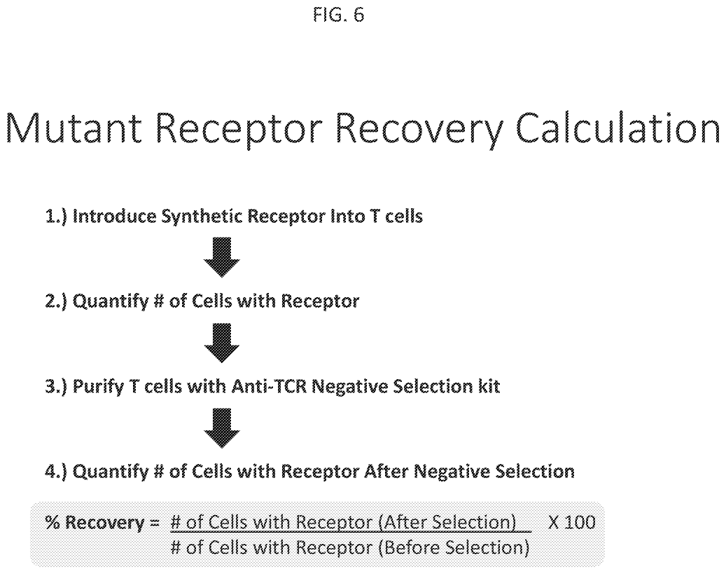

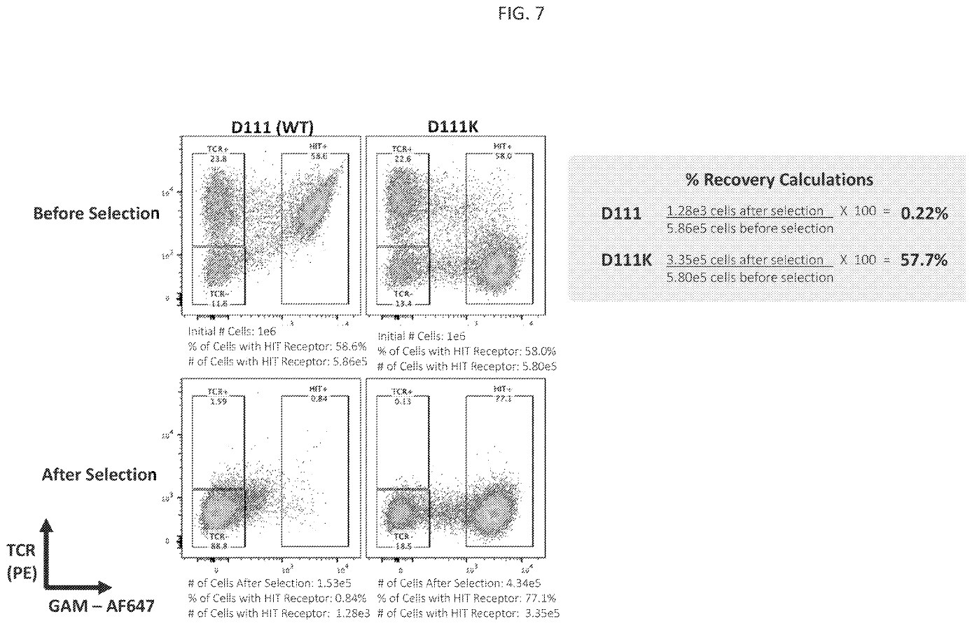

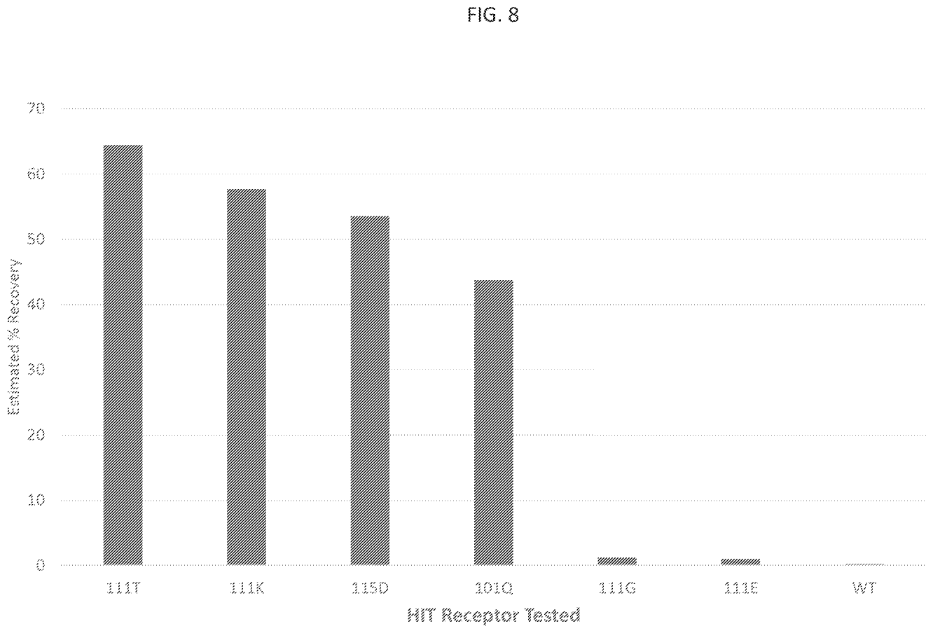

A “useful” mutation will typically have at least a 10% recovery rate. In comparison, the wild-type receptor design has a <1% recovery rate. Negative selection using a Miltenyi Biotec CliniMACS® TCRα/β Kit is performed as follows: Cells are counted using an automated cell counter. T cells are spun down and resuspended in a buffer comprised of PBS (pH~7.4), 0.5M EDTA, and 2% BSA (also referred to as MACS Buffer). Cells are incubated with biotin-conjugated anti-TCR antibody (in this instance, clone BW242/412) for 10 minutes at 4 degrees C. followed by washing of cells and resuspension in MACS buffer. Cells are then incubated with anti-biotin magnetic beads for 15 minutes at 4 degrees C., washed and again res-suspended in MACS buffer. Processed cells are separated in a magnetic column (Miltenyi MS 130-042-201) according to manufacturer's instructions. Cells that flow through the column (cells that are not bound by the anti-TCR antibody) are collected, counted, and assessed via flow cytometry. An example of such an assay is provided in FIG. 6. FIGS. 7 and 8 illustrate recovery for WT vs mutant D111K forms and recovery with various mutations, respectively. For example, as shown in FIG. 8, mutating position 111 of TCR beta chain to T, KI, D, or Q result in a binding profile providing a useful recovery rate compared to mutation to G or E.

### Nucleic Acid Encoding a Variant Beta Constant Region

In a further aspect, the disclosure further provides a nucleic acid encoding a variant beta chain constant region as provided herein and vectors comprising such nucleic acids. In some embodiments, the vector is a cloning vector, such as a plasmid or viral cloning vector. In some embodiments, the vector is an expression vector. In some embodiments, the vector is a viral vector, such as a lentiviral vector or adeno-associated viral vector (AAV).

In some embodiments, a nucleic acid encoding a variant beta chain constant region can be introduced into any type of host cell including bacterial cells, yeast cells, mammalian cells and the like. In some embodiments, the nucleic acid is dsDNA. In other embodiments, the nucleic acid is ssDNA.

In some embodiments, a viral vector, such as a lentiviral vector or adeno-associated viral vector (AAV) may be used to introduce a nucleic acid encoding a variant constant region of the present disclosure into an immune cell, e.g., a T-cell that expresses an HLA-dependent or HLA-dependent TCR. In some embodiments, a gamm-retrovirus, transposon, or recombinase-based system is used to express a variant beta chain constant region as described here. In some embodiments the T-cell is engineered, e.g., by gene editing, to prevent expression of an endogenous TCR alpha chain.

In some embodiments, a cell that expresses a variant beta chain constant region of the present disclosure is generated by gene editing, e.g., using CRISPR/Cas of an endogenous locus of a cell, e.g., a T cell or an immune precursor cell. As understood in the art, alternative systems, TALENs or zinc finger nuclease editing systems, homing endonucleases or Meganucleases may also be employed for gene editing.

### TCR Proteins Comprising a Variant TCR Beta Constant Region

In an additional aspect, the disclosure provides TCR proteins comprising a variant TCR beta constant region as described herein; and genetically modified cells, e.g., an immune cell such as a T cell, that express such proteins.

A TCR that comprises a variant beta chain constant region of the present disclosure can be any type of engineered TCR, including recombinant TCRs engineered to contain additional and/or modified domains compared to wildtype TCR proteins. In some embodiments, the TCR protein is a recombinant TCR protein heterodimer comprising a beta chain having a variant constant region of the present disclosure and an alpha chain. In some embodiments the alpha chain is native wildtype alpha chain produced by a human. In some embodiments, the alpha chain is an engineered recombinant alpha chain. In some embodiments, a recombinant TCR comprises a beta chain comprising a variant constant region as described herein and a delta chain. In some embodiments, the antigen binding chain of a TCR comprising the variant beta chain is capable of forming a trimer or oligomer with one or more identical or different constant domains and a TCR alpha chain. In some embodiments, the variable region of a beta chain comprising a variant constant region of the present disclosure is genetically modified relative to a native beta variable region occurring in nature.

In some embodiments, a TCR protein comprising a variant beta chain constant region as described herein is an HLA-independent TCR protein (described, e.g., in U.S. Pat. Appln. Publication No. 20200368283, which is incorporated by reference.

A genetically modified cell that expresses a TCR protein comprising a variant beta chain constant region as described herein is typically an immune cell, such as a T cell, or in some embodiments, or a natural killer T (NKT) cells. In some embodiments, the T cell is an effector T cell. In alternative embodiments, a T cell is a memory T cell, e.g., a central memory cell or effector memory cell. In some embodiments, the T cell is a regulatory T cell. In some embodiments, the cell is a hematopoietic stem cell or an induced pluripotent stem cells that can give rise to an immune cell, e.g., a T cell.

Preparation of a T cell Population Depleted of Cells Expressing an Endogenous TCR.

In a further aspect, a population of T cells that has been engineered to express a variant beta chain constant region provided by the present disclosure may be further subjected to screening to reduce the number of T cells that express an endogenous TCR, e.g., for use in allogeneic administration to avoid or reduce the likelihood of graft vs host complications.

In some embodiments, gene editing, e.g, CRISPR/Cas-9, can be used to integrate a transgene encoding a desired TCR-alpha chain at the TRAC locus while simultaneously disrupting expression of the endogenous TCR-alpha chain. Alternatively, the TRAC locus can be edited to disrupt expression of endogenous TCR-alpha chain and a transgene encoding a desired TCR-alpha chain can be separately introduced into the cell. In some embodiments, a nucleic acid encoding a variant beta constant region as described herein is introduced into a T cell that is gene edited to disrupt expression of endogenous beta chain to reduce mispairing. However, in some embodiments, a variant beta constant region of the present disclosure is expressed by an expression vector, e.g., a viral expression system such as a lentiviral or adeno-associated viral expression system, or a gamma-retrovirus, transposon, or recombinase-based system without disrupting the endogenous beta chain. The level of mispairing can then be assessed, e.g., quantified, using an antibody that binds to the endogenous beta chain, but not the variant beta chain. In typical embodiments, cells that express endogenous beta chain that is capable of assembly with the transgenic TCR alpha chains are removed from the population. Unedited cells that continue to express endogenous TCR are also removed. Because TCRs can exhibit different levels of mispairing, in some embodiments, a pool of TCRs expressing a variant beta chain constant region as described herein can be used to screen a pool of TCRs to identify receptors that are less prone to mispairing.

As understood in the art, although introducing a nucleic acid encoding a TCR comprising a variant beta chain as described herein may be efficient, T cells expressing endogenous TCRs may nonetheless be present in the population. Such a population may be depleted of those T cells expressing endogenous alpha beta T cell receptors by employing the antibody that binds to wild-type beta constant region epitope, but not the variant constant region for screening. Thus, for example, a BW242/412 antibody can be used to bind to T cells expressing endogenous TCR having a wildtype beta chain constant region. Such T cells can then be identified, e.g., using a fluorescently labeled BW242/412 antibody and removed from the population, e.g., by FACS. In some embodiments, depletion results in a population where less than 2%. In some embodiments, depletion can result in less than 1% of the cells express endogenous TCR. In some embodiments, multiple rounds of purification are performed.

### Pharmaceutical Compositions

In another aspect, the disclosure provides pharmaceutical compositions comprising immune cells genetically modified to express a TCR comprising a variant beta constant region as described herein and a pharmaceutically acceptable diluent, carrier or excipient. In some embodiments, the immune cells are T cells from a population of T cells depleted of cells expressing an endogenous TCR.

Pharmaceutical compositions comprising engineered T cells of the present invention may comprise buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; and preservatives.

In some embodiments, an immune cell expressing a TCR comprising a variant beta constant region of the present disclosure is used in a method of killing cancer cells or treating cancer in a subject, comprising administering to the subject a therapeutically effective amount of a the immune cells. In some embodiments, the cancers cells express a target antigen. In some embodiments, the TCR is HLA-independent. In some embodiments, the cancer is a blood or hematological cancer. Exemplary cancers include, but are not limited to, breast cancer, prostate cancer, ovarian cancer, glioblastoma, cervical cancer, skin cancer, pancreatic cancer, colorectal cancer, bladder cancer, endometrial cancer, renal cancer, liver cancer, brain cancer, lymphoma, leukemia (for example, acute myeloid leukemia), myeloma, lung cancer, and the like. It is understood that the methods provided herein can also be used to target circulating cancer cells, for example, cells shed by a solid tumor into the bloodstream of a subject.

In some embodiments, immune cells, e.g., T cells, are obtained from the subject and modified to express a TCR comprising a beta chain constant region as described herein, e.g., an antigen-specific TCR, or HLA-independent T cell, prior to administering the modified T cells to the subject. In some embodiments, the immune cells are tumor-infiltrating lymphocytes.

In some embodiments, an immune cell expressing a TCR comprising a variant beta constant region of the present disclosure can be used for the treatment of an infectious disease, an autoimmune disease or other inflammatory disease in a subject. In some embodiments, the T cells are regulatory T cells.

In some embodiments, an immune cell expressing a TCR comprising a variant beta chain constant region as described herein is employed in allogeneic applications for adoptive cell therapy.

As used herein, the term “subject” refers to an individual. In the present application, a subject that is treated with a TCR comprising a variant beta chain constant region is typically a human. Such a subject can be an adult subject or a pediatric subject. Pediatric subjects include subjects ranging in age from birth to eighteen years of age.

Although a human subject is typically treated with immune cells comprising a modified TCR of the present disclosure, the term “subject” includes a non-human primate (e.g., a monkey or chimpanzee). Thus, for example, non-human primate beta chain polypeptides that comprise a BW242/412 epitope can also be modified to abrogate binding of antibody as described herein.

### Methods of Screening/Library Preparation

In a further aspect, the invention provides a method of identifying candidate residues in a TCR constant regions, e.g., TCR beta chain, that abrogate binding of an antibody to a TCR constant chain-specific epitope, e.g., beta chain-specific epitope, and libraries for practicing such methods. Thus, in one embodiment, the disclosure provides a library of cells, e.g., T cells that are engineered to express TCR beta constant domains comprising the extracellular portion of the domain. Mutations are introduced throughout the constant domain, by systematically mutating residues across the domain. In some embodiments, the library is generated by engineering a population of T cells at the human TRAC locus by gene editing, e.g., CRISPR/Cas editing, to express a library of TCRs. In some embodiments, the TCR is an HLA-dependent TCR. Alternatively, the receptor may be an HLA-independent receptor. Gene editing can also be performed by alternative gene editing techniques such as TALENs or zinc finger nuclease editing techniques.

Libraries can be generated using various mutagenesis techniques. In some embodiments, mutations are introduced into a HIT receptor that comprises a TCR beta chain constant region to be mutated. For example, a HIT receptor (Mansilla-Soto, Eyquem et al Nature Medicine, 2022) is generated by remodeling the T cell receptor alpha (TRAC) locus and replacing the variable chains of the TCR with the heavy and light chain of a scFv. This strategy yields T cells that lack their endogenous TCR but acquire the ability to engage cell-surface targets through their CD3 complex. This receptor is more sensitive to low antigen densities than the Chimeric Antigen Receptor (CAR).

Mutagenesis can be performed using any technique or combinations. In some instances, alanine scanning mutagenesis is performed. In some instances, saturation scanning mutagenesis, substituting each residue with every other residue, or a variation of saturation mutagenesis that introduces fewer alternative amino acids at each site is employed. In some embodiments, amino acid substitutions at one or more sites can be selected based on prediction matrices that score the favorability, e.g., for BLOSUM matrices, such as the BLOSUM80 matrix and the PAM matrices.

A library can be screened against an antibody that specifically binds to the constant region of a TCR beta chain. Populations of cells can be obtained for comparison that include a control “baseline” population (i.e., a population from the library of cells prior to antibody binding), a population that binds to the antibody of interest and a population that does not bind to the antibody of interest. Cells not bound by the antibody are further be divided based on whether or not they express the HIT receptor.

One of skill understands that any library of receptor variants, e.g., having mutations in an extracellular domain, can be generated as detailed above. Such libraries can be screened against an antibody that specifically binds to an epitope of interest to identify mutations that abrogate antibody binding to the update but otherwise preserves receptor function.

As understood by one of skill in the art, a library of receptors can be

### METHODOLOGY AND TECHNIQUES

The following techniques can be employed for removing an anti-TCR antibody epitope from the extracellular domain of a TCR beta chain constant region.

### Library Design:

### Constant Domain Mutagenesis Strategy:

The overall objective of this screening process was to disrupt the binding site of the BW242/412 antibody without disrupting the performance of the HIT receptor. To accomplish this, a library of single AA mutations was introduced into the extracellular constant region of TRBC in the HIT receptor.

Individual residues within the TRBC domain of a HIT receptor were systematically mutated. These mutations included:
Deleting the residue;
Substituting the residue with alanine;
Substituting the residue with amino acids predicted conserve the HIT receptor's structure and function (based on BLOSUM scores for that position);
Substituting the residue with residues seen in TRBC domains of other species; and a synonymous mutation (as a control). Further details are as follow:

In one instance, substitutions were introduced systematically across the extracellular domain of TRBC using two processes:

### 1. BLOSUM Directed Mutagenesis/Alanine Scanning:

At each residue, alanine was substituted. Additionally, four other amino acid substitutions that were predicted to be non-disruptive based on scores from the BLOSUM80 matrix were systematically introduced. Five substitutions from the BLOSUM80 matrix were introduced into residues that were originally alanine.

TABLE 1
Substitution Matrix

| Original AA | A | R | N | D | C | Q | E | G | H | I | L | K | M | F | P | S | T | W | Y | V |
|---|---|---|---|---|---|---|---|---|---|---|---|---|---|---|---|---|---|---|---|---|
| Substituted | S | K | D | N | T | E | Q | N | Y | V | M | R | L | Y | K | T | S | Y | F | I |
| AAs | G | Q | Q | E | V | R | D | S | Q | L | I | Q | I | L | S | N | V | F | H | L |
| | T | H | H | Q | I | H | K | D | R | M | V | E | V | M | R | Q | N | L | W | M |
| | C | N | K | K | L | K | H | Q | N | F | F | N | Q | W | D | E | R | M | Q | T |
| | V | A | A | A | A | A | A | A | A | A | A | A | A | A | A | A | A | A | A | A |

Based on this matrix, a residue with a “D” would be substituted with “N”, “E”, “Q” “K”, or “A”.

### 2. Homology Informed Substitutions:

Additionally, 38 homologous protein sequences (list below) from mammals were aligned with the TRBC sequence and the frequency of each substitution at each residue (see attached excel file—Homologous Sequence Alignment) was determined. All homologous substitutions were included in the generated library. For example, D111 has homologous substitutions for “A”, “E”, “P”, “N”, “T”, and “Q”.

Therefore, the final mutations made at D111 using both strategies would be “N”, “E”, “Q”, “K”, “A”, “P”, “T”, and “Q”. The accession numbers for sequences used for homology assessment are provided in Table 2.

TABLE 2
Sequences Used For Homology Assessment

| | |
|---|---|
| 1. | 0A5H1ZRT1_HUMAN |
| 2. | 0A2J8QUE2_PANTR |
| 3. | 2PP94_PONAB |
| 4. | 0A2J8V300_PONAB |
| 5. | 0A0G2JMB4_HUMAN |
| 6. | 0A5H1ZRR3_HUMAN |
| 7. | 0A0D9R5B1_CHLSB |
| 8. | 0A2I3SCJ3_PANTR |
| 9. | 7MMZ5_MACMU |
| 10. | 0A2R8ZDC5_PANPA |
| 11. | 0A2I2YRH4_GORGO |
| 12. | 0A5E4AQB3_MARMO |
| 13. | 1PSA6_MYOLU |
| 14. | 0A4W2ERJ9_BOBOX |
| 15. | 1MJJ6_BOVIN |
| 16. | 1MUR8_BOVIN |
| 17. | 3MXJ1_BOVIN |
| 18. | 8IAU9_9CETA |
| 19. | 0A452E0J8_CAPHI |
| 20. | 0A452E0R0_CAPHI |
| 21. | 1PEQ3_CANLF |
| 22. | 0A452ERL6_CAPHI |
| 23. | 0A2K6GVW3_PROCO |
| 24. | 0A2K6GVV9_PROCO |
| 25. | 2HP35_AILME |
| 26. | 1LIB9_AILME |
| 27. | 2I8R3_AILME |
| 28. | 2HP36_AILME |
| 29. | 0A4W2HJF4_BOBOX |
| 30. | 1MJB8_BOVIN |
| 31. | 0A673SS53_SURSU |
| 32. | 0A673SS69_SURSU |
| 33. | 0A485NS05_LYNPA |
| 34. | 3YAT2_MUSPF |
| 35. | 0A3P4NJG5_GULGU |
| 36. | 0A2K5PVI3_CEBCA |
| 37. | 0A2K5PVI6_CEBCA |
| 38. | 0A2K6TGF3_SAIBB |

The library was divided into 3 tiles for synthesis:
Tile 1: Mutations in residues 1-50
Tile 2: Mutations in residues 51-100
Tile 3: Mutations in residues 101-150
   Each library was cloned into a HIT receptor backbone, sequenced, and produced as an adeno-associated virus (AAV).

### T Cell Engineering:

HIT libraries were introduced into the TRAC locus of primary human T cells using CRISPR-Cas9 editing (unsorted library). Since T cells experience allelic exclusion at the TRAC locus, each T cell would express only one member of our HIT library.

Screen: A bead-based TCR isolation kit was employed to separate the unsorted library into two populations: T cells that were bound by TCR antibodies (column bound) and T cells that were not bound by the TCR antibodies (unbound). The unbound sample was then stained with an antibody specific for the HIT receptor and sorted via FACs into 3 populations: T cells with undetectable HIT expression (HIT low), T cells with intermediate HIT expression (HIT mid), and T cells with normal HIT expression (HIT high).

Library Quantification: To determine the abundance of each receptor variant in these populations, RNA was harvested from T cells, the library region amplified using RT-PCR, and the frequency of each library member quantified using next generation sequencing. This process was conducted with samples from the unsorted library, the column bound sample, the HIT low sample, and the HIT high sample. Receptor enrichment/depletion was determined by comparing the abundance of a receptor in an experimental condition to the abundance of that receptor in the unsorted library. Receptors that were enriched in the HIT high sample and depleted in the column bound sample were selected for validation.

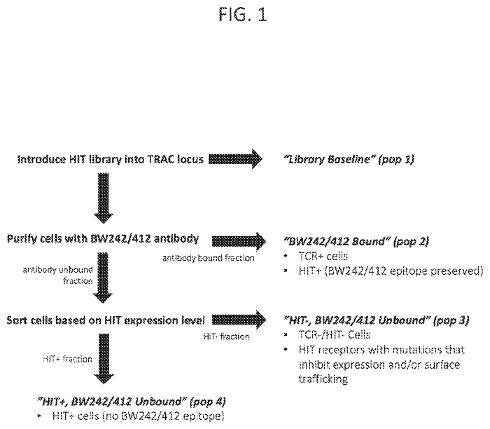

FIG. 1 depicts the screening strategy summarized above. T cells were engineered to express a library of HIT receptors, expressed from the TRAC locus) via AAV and CRISPR/Cas editing. A sample of this population was then preserved as a baseline for the screen (pop 1). After transduction, the library was sorted by BW242/412 binding.

Binding conditions employed to determine BW242/412 binding in the library screen were based on the protocol provided for the TCR purification kit (Mitenyi). More specifically the library of T cells was resuspended in MACs buffer (PBS, 0.5% FBS, 2 mM EDTA) at 10e7 cells/80 μL buffer. Thirty L of biotin-conjugated BW242/412 antibody was added per 10e7 cells. Cells were incubated for 10 minutes at 4° C. Cells were washed with MACS buffer and resuspended at 10e7 cells/80 buffer. Thirty μL of anti-biotin beads were added per 10e7 cells. Cells were washed and resuspended in MACS buffer. The screening conditions are stringent. When such conditions are employed for purification, greater than 98% of antibody-bounds cells are removed.

Cells bound by BW242/412 were isolated (population 2) while cells not bound by BW242/412 were further sorted by HIT expression via Fluorescence-Activated Cell Sorting (pop 3 and of mutant in D111 in BW242/412-Unbound, HIT+ fraction (pop 4). The frequencies of each mutant HIT receptor in a given population was determined by RNA-seq. These frequencies were then normalized to the frequency of the mutant in the library baseline (pop 1).

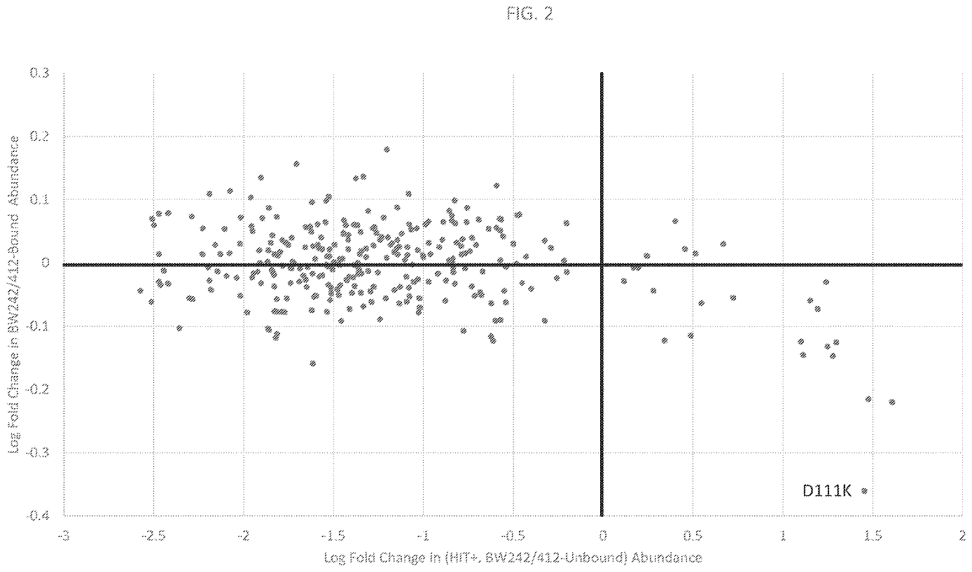

The relative abundance of mutants in the BW242/412-bound fraction (pop 2) vs. the abundance in the HIT+BW242/412-unbound fraction (pop 4) is shown in FIG. 2. Each dot represents a single AA substitution. The screen identified several promising mutations with D111K exhibiting the most notable combination of HIT expression and resistance to BW242/412 depletion.

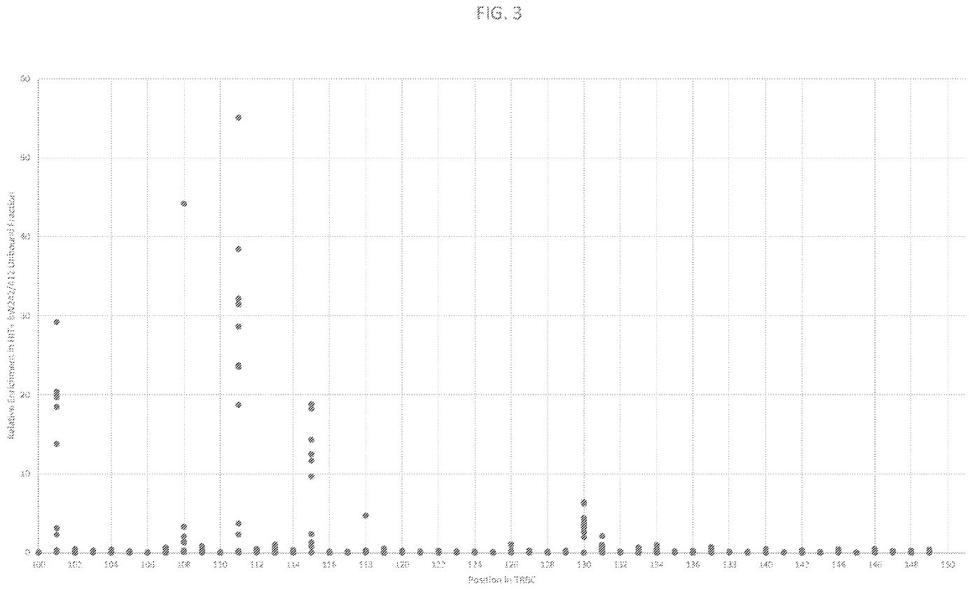

FIG. 3 provides data showing the enrichment of mutant in BW242/412-Unbound, HIT+ fraction (pop 4) relative to library baseline (pop1) by location in TRBC. Each do represents a single amino acid substitution.

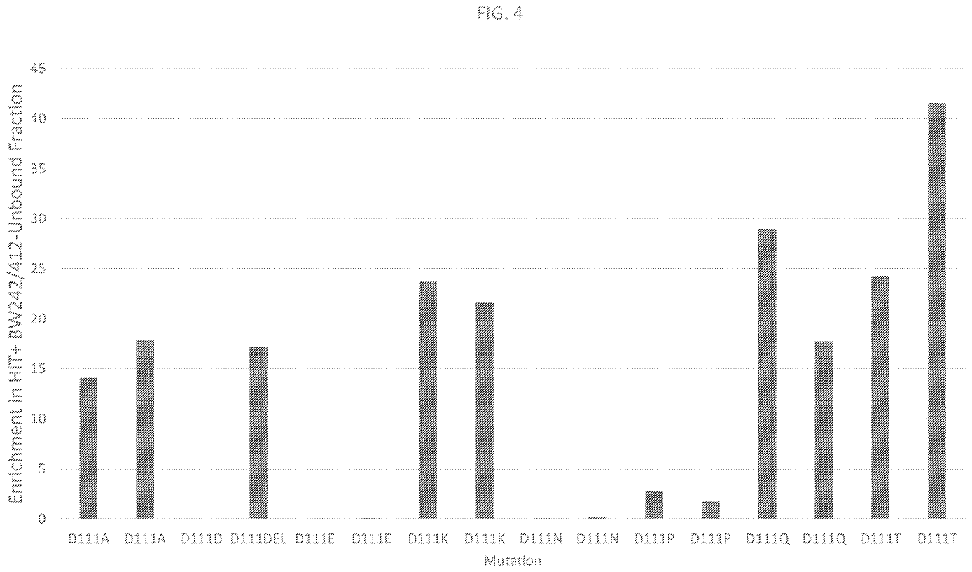

FIG. 4 shows the enrichment of mutants at D111 in BW242/412-Unbound, HIT+ fraction (pop 4) relative to library baseline (pop 1). Previous studies (Kierkels et al, 2021 showed that the BW242/412-epitope can be eliminated by substituting two murine residues ((D111G) combined with E108K or T110P)). BW242/412 binding does not appear to be affected by a D111E mutations. Conversely, the D111K mutation, which creates a positive charge at the residue, is sufficient to eliminate binding. This suggests that D111K abrogates binding of BW242/412 to the TCR beta chain epitope by disrupting charge interactions.

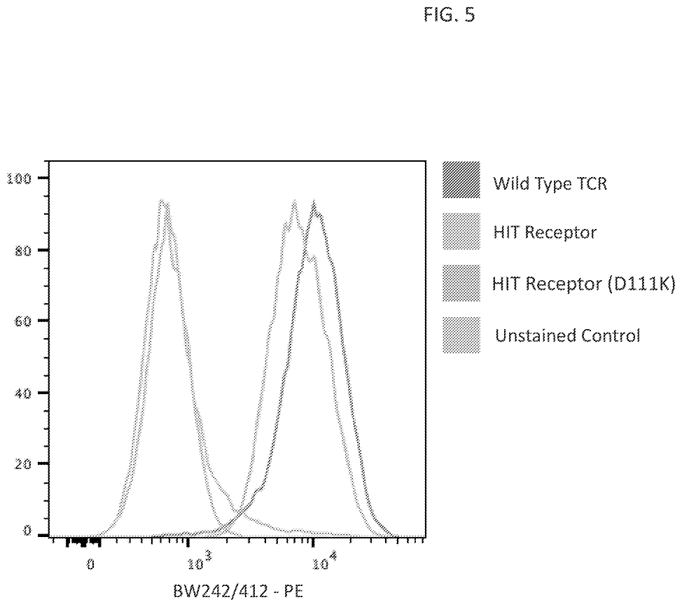

FIG. 5 provides data showing that D111K exhibits minimal BW242/412 binding. In this assay, binding was assessed as described above. T cells were engineered to express a wild-type HIT receptor or a HIT receptor with the D111K mutation from the TRAC locus via CRISPR/Cas-9 editing. Engineered T cells and unmodified wild-type T cells were stained with PE-conjugated BW242/412 at 4° C. for 15 minutes in a buffer comprised of PBS (pH~7.4), 0.5M EDTA, and 2% FBS. Additionally, an unstained control condition was prepared with unmodified wild-type T cells. Signal from the PE-BW242/412 antibody was then assessed via flow cytometry.

FIGS. 6-8 illustrate calculation of recovery rates of mutated TCR beta chain polypeptides. In particular, FIG. 6 provides a general calculation of recovery rate of a mutant TCR beta polypeptide.

FIG. 7 provides an example of receptor recovery for 2 HIT receptors, wild-type and D111K. Purification was performed as described above. For this experiment, T cells were edited to express a WT HIT receptor or a HIT receptor with the D111K mutation. The edited T cells were then stained with antibodies for the scFv attached to the HIT receptor (GAM—Y axis) or with the BW242/412 anti-TCR antibody (x-axis). Thus, cells that express a HIT receptor are on the right side of these flow plots. Before selection, ~58% of both conditions contain cells with a HIT receptor. For each condition, 1e6 total T cells were then processed with the anti-TCR negative selection kit (as described above). In this kit, cells that are bound by the TCR antibody are retained in a column, while cells that are not antibody-bound flow through the column. After negative selection, 1.5e5 total cells were recovered from the WT HIT cell condition and 4.3e5 total cells were recovered from the HIT D111K condition. Flow cytometry was then used to determine the % of cells in this population that express a HIT receptor. With this information, the number of cells with a given receptor can be estimated before and after negative selection. Here, 58% of the initial 1e6 cells in the D111K condition (prior to selection) expressed a HIT receptor—thus 5.80e5 HIT+ cells before selection. After selection, 4.34e5 total cells were recovered and 77.1% were HIT positive, so we determine that 3.35e5 HIT+ cells were recovered. Therefore, the % recovery for D111K would be 57.7%.

FIG. 8 illustrates recovery rates, determined as indicated above, for various mutations at position 111. Based on these data, the 111T, 111K, 115D, and 102Q mutations can be selected for mutating TCR beta chains for generating T cell populations for immunotherapy.

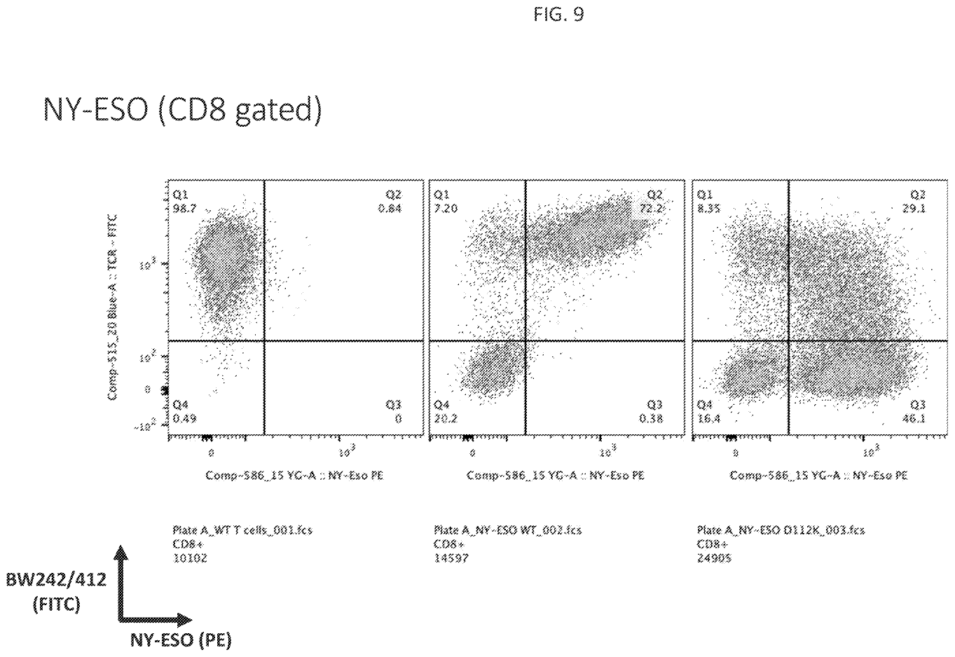

In a further experiment to assess D111K modification of a TCR, T cells were edited to express an unmodified NY-ESO TCR (wild-type) or an NY-ESO TCR with the D111K modification made in the TRBC constant region via CRISPR/Cas-9 editing. BW242/412-binding (FITC) was assessed by flow cytometry in CD8+ T cells and compared with an NY-ESO dextramer stain (PE) to confirm TCR receptor expression. Additionally, unmodified T cells were stained as a control. In this experiment (FIG. 9), the NY-ESO D111K TCR exhibited markedly reduced anti-TCR antibody (BW242/412) binding. In the NY-ESO dextramer-positive population, 60% of the population was not stained by the anti-TCR antibody, vs 0% in WT. As a results the recovery of NYESO D111K post purification is 80%.

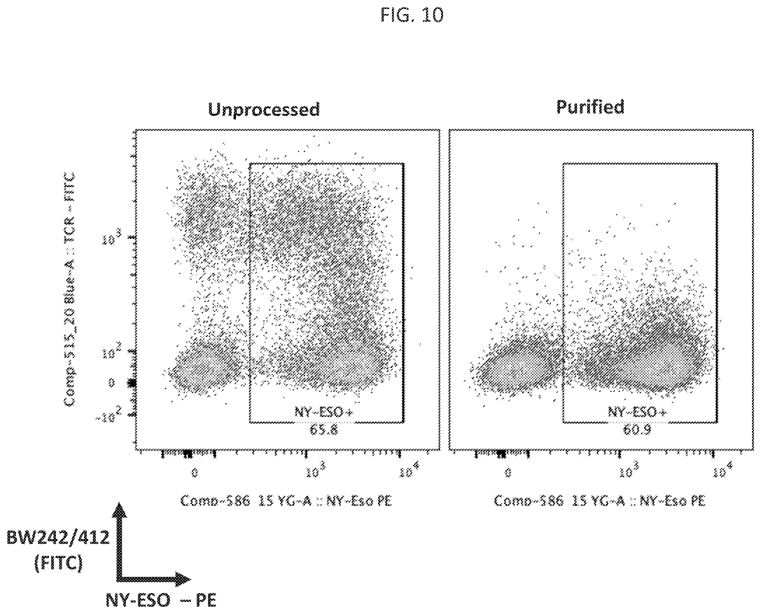

T cells (1e6 cells) edited to express the NY-ESO D111K TCR were processed via the Miltenyi CliniMACs negative selection kit (as previously described). The unpurified and purified populations were then compared via flow cytometry after being stained with the BW242/412 antibody and NY-ESO TCR dextramer (FIG. 10). Purification resulted in a population of T cells in which less than 1% retained an endogenous TCR.

### Saturation TRBC Mutagenesis Analyses

Saturation mutagenesis was also performed to comprehensively catalog conservative and non-conservative substitutions at positions G101, D111, and P115. These libraries were introduced into T cells and sorted into two groups based on high and low TCR antibody BW242/412 binding, i.e., whether the substitutions were enriched in the BW242/412-unbound (antibody disrupted binding) vs. bound population (antibody binding analysis preserved.

Site saturation mutagenesis was performed by synthesizing pools of oligonucleotides encoding TRBC residues 101-150 with degenerate nucleotides specified at bases encoding the residue of interest. Separate pools were synthesized with mutations introduced at either G101, D111, or P115. Each pool was then cloned into a HIT receptor backbone, sequenced, and produced as an AAV.

HIT libraries were introduced into the TRAC locus of primary human T cells using CRISPR-Cas9 editing. Transduced HIT+ T cells were then sorted based on BW242/412 binding via Fluorescence-Activated Cell Sorting (FACS). More specifically, transduced T cells were:
Resuspended in MACs buffer at 10e7 cells/1 mL buffer;
Stained with an antibody specific for the HIT receptor and incubated for 10 min at 4° C.;
Washed with 10 mL of MACs buffer then resuspended at 10e7 cells/1 mL buffer;
Incubated with mouse serum (2% of sample volume);
Washed with 10 mL of MACs buffer then resuspended at 10e7 cells/1 mL buffer;
Stained with PE-conjugated BW242/412 (1 uL antibody/1e6 cells) and incubated for 15 min at 4° C.;
Washed twice 10 mL of MACs buffer then resuspended at 10e7 cells/1 mL buffer; and
Sorted into (HIT+| BW242/412-bound) and (HIT+| BW242/412-unbound) populations.

During sorting, gating for the (HIT+BW242/412-unbound) was set based on a stained sample of non-transduced TRAC-edited cells.

We then quantified the relative abundance of each codon in both low and high binding to determine whether a given mutation preserved antibody binding or reduced antibody binding. To determine the abundance of each receptor variant in these populations, we harvested RNA from T cells, amplified the library region using RT-PCR, and quantified the frequency of each library member using next generation sequencing. This process was conducted with samples from before and after the sorting process.

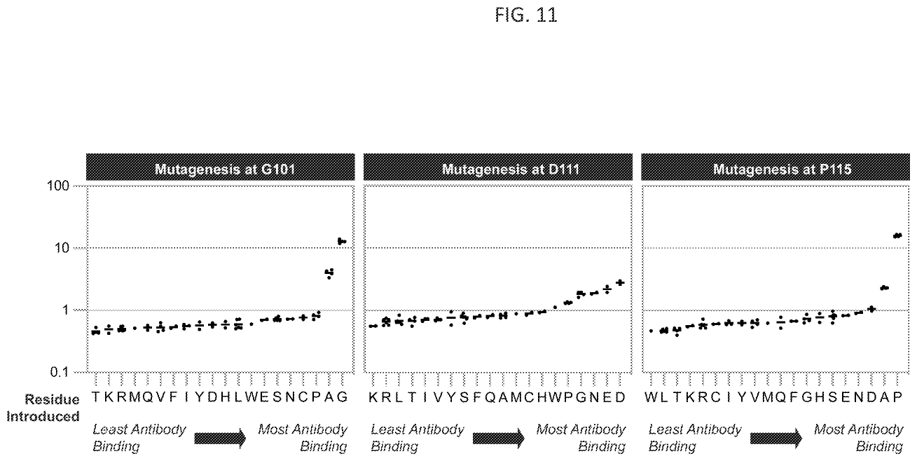

Results are shown in FIG. 11. In the panels shown in FIG. 11, each dot represents a single codon introduced at the target residue. Dots are grouped by the AA they encode and sorted based on the average enrichment score. Values below 1 represent substitutions which are enriched in the BW242/412-unbound population (antibody binding disrupted), while values above 1 represent substitutions which are enriched in the BW242/412-bound population (antibody binding preserved).

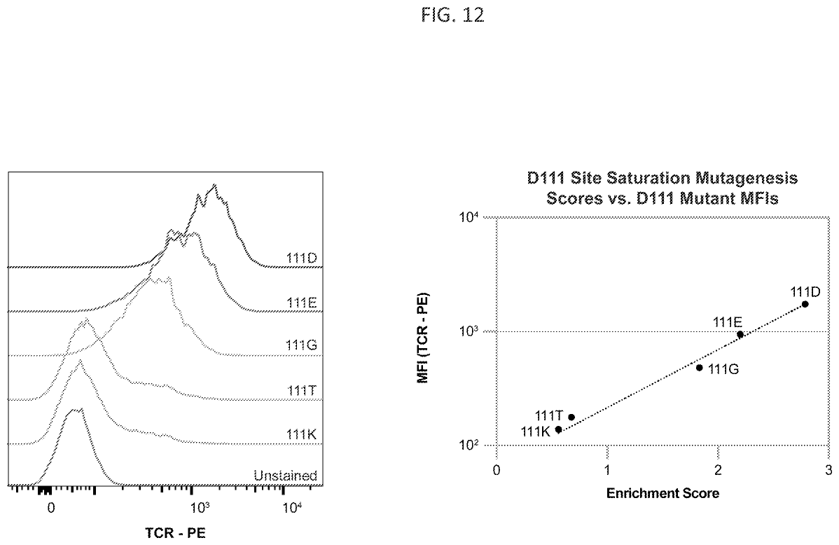

In FIG. 12, T cells were transduced to express HIT receptors with 5 different amino acids at TRBC D111. BW242/412 binding was then quantifying by measuring the mean fluorescence intensity (MFI) of HIT+ cells stained with PE-conjugated BW242/412. These results were then compared to enrichment scores calculated during the site saturation mutagenesis screen at TRBC D111. These results provide further evidence that pooled site-saturation mutagenesis screens accurately predict antibody binding modulation.

This screening methodology track all possible substitutions at a given residue. These experimental data establish that any mutations at G101, D111, or P115 reduces bW242/412 antiboding binding when compared to the wild-type sequence.

TRBC Mutagenesis Allows for the Isolation of TRAC-Edited Cells that Exhibit Minimal Levels of Mispairing:

T cells can be re-programmed to target a specific antigen by engineering them to express anew (transgenic) TCR. However, co-expression of multiple TCRs in a single T cell can lead to mispairing between endogenous and transgenic TCR chains. TCR mispairing reduces the cytotoxicity of T cells engineered to express a transgenic TCR. Additionally, T cells with mispaired TCRs may target healthy tissue leading to the development of graft-versus-host disease. The current standard for minimizing mispairing in T cells engineered to express a transgenic TCR is to disrupt by gene editing both the endogenous TCR-alpha and TCR-beta chains. However, performing simultaneous gene editing at multiple loci can reduce cell viability and lead to genomic translocations.

CRISPR/Cas-9 (and other gene editing technologies) can be used to introduce a transgene at the TRAC locus while simultaneously disrupting expression of the endogenous TCR-alpha chain. If this method is used to introduce a transgenic TCR, mispairing can occur between the transgenic TCR-alpha chain and the endogenous TCR-beta chain.

If a transgenic TCR with an unmodified TCR-beta constant domain is introduced at TRAC, BW242/412 will bind to both mispaired and fully transgenic TCRs. However, if a transgenic TCR-beta chain with the BW242/412 epitope ablated [via the D111K mutation or any other method] is used, mispaired TCRs will bind BW242/412, while fully transgenic TCRs will not.

Previous studies have established that interactions between TCR-alpha and beta variable domains can influence TCR heterodimerization and surface expression. Data generated from TRAC-edited cells transduced with a D111K modified transgenic-TCR (specific for NY-ESO) show that some edited cells have consistently high levels of TCR mispairing while other edited cells have consistently low levels of TCR mispairing. Since only mispaired TCR bind BW242/412 in this system, cells with low levels of mispairing can be isolated via antibody-based negative selection. After negative TCR selection, non-mispaired NYESO TCR T cells are enriched without the need for endogenous TCR beta KO.

Primary human T cells were edited at TRAC and TRBC via CRISPR/Cas9 and transduced to express an NY-ESO transgenic TCR with either an unmodified TCR beta constant domain (NY-ESO WT TRBC) or a TCR beta constant domain with the D111K mutation (NY-ESO TRBC D111K) from the TRAC locus. Edited CD8⁺ cells were then stained with FITC-conjugated anti-TCR antibody (BW242/412) and PE-conjugated NY-ESO dextramer and analyzed via flow cytometry.

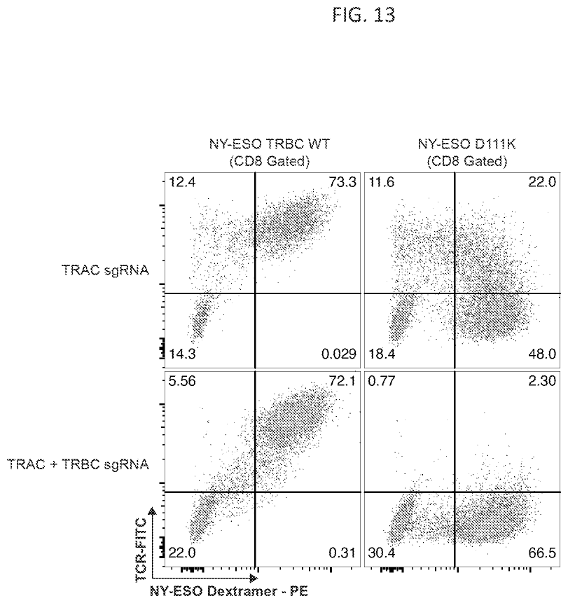

As shown in in FIG. 13, a portion of TRAC-edited CD8⁺ cells engineered to express NY-ESO D111K (upper right flow plot) are stained by both BW242/412 and NY-ESO dextramer. However, this population does not appear in cells edited at TRAC and TRBC that express NY-ESO D111K (lower right flow plot).

These results indicated that that TRAC edited cells that are stained by both BW242/412 and NY-ESO dextramer express a mispaired TCR which consists of a transgenic NY-ESO TCR alpha chain and an endogenous TCR beta chain.

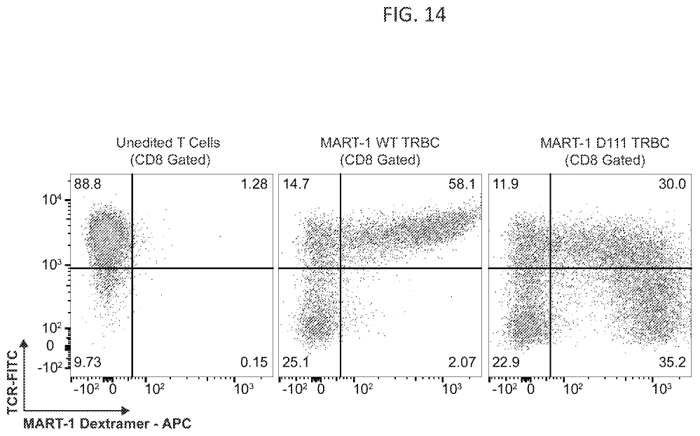

Primary human T cells were edited at TRAC via CRISPR/Cas9 and transduced to express a MART-1 transgenic TCR with either an unmodified TCR beta constant domain (MART-1 WT TRBC) or a TCR beta constant domain with the D111K mutation (MART-1 TRBC D111K) from the TRAC locus. Edited CD8⁺ cells were then stained with FITC-conjugated anti-TCR antibody (BW242/412) and APC-conjugated MART-1 dextramer and analyzed via flow cytometry. FIG. 14 shows that in the MART-1 WT TRBC condition, all dextramer positive cells (cells which express a transgenic TCR) are stained by BW242/412, while in the MART-1 TRBC D111K condition dextramer positive cells exhibit a range of BW242/412 levels.

All publications, patent applications, and accession numbers mentioned in this specification are herein incorporated by reference to the same extent as if each individual publication or patent application was specifically and individually indicated to be incorporated by reference for the material for which it is cited.

TABLE of Illustrative Sequences

SEQ ID NO: 1 UniProtKB-P10850-T cell receptor beta constant 1 (176 aa);
the extracellular domain is italicized.
DLNKVFPPEVAVFEPSEAEISHTQKATLVCLATGFFPDHVELSWWVNGKEVHSGVSTDPQ
PLKEQPALNDSRYCLSSRLRVSATFWQNPRNHFRCQVQFYGLSENDEWTQDRAKPVTQIV
SAEAWGRADCGFTSVSYQQGVLSATILYEILLGKATLYAVLVSALVLMAMVKRKDF
SEQ ID NO: 2 UniProtKB-A0A5B9--T cell receptor beta constant 2 (178 aa)
DLKNVFPPKVAVFEPSEAEISHTQKATLVCLATGFYPDHVELSWWVNGKEVHSGVSTDPQ
PLKEQPALNDSRYCLSSRLRVSATFWQNPRNHFRCQVQFYGLSENDEWTQDRAKPVTQIV
SAEAWGRADCGFTSESYQQGVLSATILYEILLGKATLYAVLVSALVLMAMVKRKDSRG
SEQ ID NO: 3 TCR beta chain C region(178 aa) UniprotKB-P0DTU4-T cell
receptor beta constant MC.7.G5
DLKNVFPPEVAVFEPSEAEISHTQKATLVCLATGFYPDHVELSWWVNGKEVHSGVSTDPQ
PLKEQPALNDSRYCLSSRLRVSATFWQNPRNHFRCQVQFYGLSENDEWTQDRAKPVTQIV
SAEAWGRADCGFTSESYQQGVLSATILYEILLGKATLYAVLVSALVLMAMVKRKDSRG

What is claimed is:

1. A nucleic acid encoding a human T Cell Receptor (TCR) beta chain comprising a mutated constant region having at least 95% sequence identity to any one of SEQ ID NOS:1, 2, or 3, wherein the constant region comprises a substitution at a position corresponding to position D111, wherein the substitution is any amino acid other than E or G.

2. The nucleic acid of claim 1, wherein the substitution is R, L, I, V, Y, S, F, M, C, or H.

3. The nucleic acid of claim 1, wherein the constant region comprises a substitution D111K, D111T, D111Q, or D111A; or the constant region comprises a deletion at the position corresponding to D111.

3. The nucleic acid of claim 1, wherein the substitution or deletion abrogates binding of the beta chain polypeptide to antibody BW242/412.

4. The nucleic acid of claim 1, wherein the substitution is D111K.

5. The nucleic acid of claim 1, wherein the substitution is D111T or D111Q.

6. The nucleic acid of claim 1, wherein the mutated constant region comprises a deletion at the position corresponding to D111.

7. The nucleic acid of claim 1, wherein the mutated constant region further comprises a substitution at at least one position corresponding to a position selected from G101, W108, P115, Q118, C130, and G131.

8. The nucleic acid of claim 7, wherein the substitution at G101 is Q, D, or N; the substitution at position at position 108 is A, R, M or L; the substitution at P115 is K, R, or D; the substitution at position 130 is V, E, L or I; and the substitution at position 131 is N.

9. A nucleic acid encoding a human TCR beta chain polypeptide comprising a mutated constant region having at least 95% sequence identity to any one of SEQ ID NOS:1, 2, or 3, wherein the constant region comprises at least one substitution at a position corresponding to position G101.

10. The nucleic acid of claim 9, wherein the substitution at G 101 is T, K, R, M, Q, V, F, I, Y, D, H, L, W, E, S, N, C, or P.

11. The nucleic acid of claim 10, wherein the substitution is Q, D, or N.

12. The nucleic acid of any one of claims 9-11, comprising at least two substitutions in the constant region.

13. A nucleic acid encoding a human TCR beta chain polypeptide comprising a mutated constant region having at least 95% sequence identity to any one of SEQ ID NOS:1, 2, or 3, wherein the constant region comprises at least one substitution at a position corresponding to position G115.

14. The nucleic acid of claim 13, wherein the substitution is W, L, T, K, R, C, I, Y, V, M, Q, F, G, H, S, E, N or D.

15. The nucleic acid of claim 14, wherein the substitution is K, R, or D.

16. The nucleic acid of any one of claims 13-15, comprising at least two substitutions in the constant region.

17. A nucleic acid encoding a human TCR beta chain polypeptide comprising a mutated constant region having at least 95% sequence identity to any one of SEQ ID NOS:1, 2, or 3, wherein the constant region comprises at least one substitution at a position corresponding to position W108, Q118, C130, or G131, wherein the substitution at position 108 is A, R, M or L; the substitution at position 130 is V, E, L or I; or the substitution at position 131 is N, wherein the substitution reduces binding of an antibody BW242/412 to the mutated constant region.

18. The nucleic acid of any one of claims 1-17, wherein the mutated constant region has at least 95% identity to SEQ ID NO:1.

19. The nucleic acid of any one of claims 1-17, wherein the mutated constant region has at least 95% identity to SEQ ID NO:2.

20. The nucleic acid of any one of claims 1-17, wherein the mutated constant region has at least 95% identity to SEQ ID NO:3.

21. The nucleic acid of any one of claims 1-20, wherein the human TCR beta chain is comprised by an HLA-independent TCR.

22. The nucleic acid of any one of claims 1-20, wherein the human TCR beta chain is comprised by an HLA-dependent TCR.

23. A host cell comprising the nucleic acid of any one of the foregoing claims.

24. The host cell of claim 23, wherein the cell is a T cell, a human embryonic stem cell or a pluripotent stem cell.

25. The host cell of claim 24, wherein the host cell is a T cell.

26. The host cell of claim 24, wherein the host cell is a T cell selected from the group consisting of a cytotoxic T lymphocyte, a regulatory T cell, and a Natural Killer T cell.

27. The host cell of any one of claims 24-26, wherein the host cell is engineered to express a transgenic TCR-alpha chain.

28. The host cell of claim 30, wherein the host cell is edited to disrupt expression of endogenous TCR-alpha chain.

29. The host cell of claim 28, wherein the transgenic TCR-alpha chain is introduced into the host cell at the TRAC locus and disrupts expression of endogenous TCR-alpha chain.

30. A population of T cells comprising a host cell of any one of claims 24-29.

31. The population of claim 30, wherein the number of T cells that express an endogenous T cell receptor beta chain constant region in a population of screened T cells is less than 5%.

32. The population of claim 30, wherein the number of T cells that express an endogenous T cell receptor beta chain constant region in a population of screened T cells is less than 1%.

33. A method of treating cancer comprising administering to a subject, a population of T cells of claim 31.

34. A method of determining the amount of mis-pairing between endogenous TCR beta chain and a transgenic T cell receptor alpha chain expressed by a population of T cells comprising a nucleic acid of any one of claims 1-20 that expresses a variant TCR beta chain, the method comprising determining the level of binding of an antibody to individual T cells in the population.

35. The method of claim 34, wherein the transgenic T cell receptor alpha chain is edited to disrupt expression of endogenous TCR-alpha chain.

36. The method of claim 35, wherein the transgenic TCR-alpha chain is introduced into the T cell at the TRAC locus and disrupts expression of the endogenous TCR-alpha chain.

37. The method of claim 34, 35, or 36, further comprising removing T cells that bind the antibody from the population.

38. A library of cells that express mutated TCR beta constant regions wherein the library comprises cells comprising different mutations of the TCR beta constant region that span the constant region domain.

39. The library of claim 38, wherein the cells are T cells edited at the human TRAC locus to express different mutations of the TCR beta chain constant region.


SEQUENCE LISTING (3 sequences)

SEQ ID NO: 1 - protein, 176 aa, Tetrahymena pyriformis
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 2 - protein, 178 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 3 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG